(19) United States
(12) Patent Application Publication
(10) Pub. No.: US 2009/0182378 A1
(43) Pub. Date: Jul. 16, 2009

(54) FLEXIBLE ROD FOR FIXING VERTEBRAE

(76) Inventor: Gil Woon Choi, Anyang-si (KR)

Correspondence Address: LEE, HONG, DEGERMAN, KANG & WAIMEY, 660 S. FIGUEROA STREET, Suite 2300, LOS ANGELES, CA, 90017, US

(21) Appl. No.: 12/029,014

(22) Filed: Feb. 11, 2008

(30) Foreign Application Priority Data
Jan. 11, 2008 (KR) 10-2008-0003516

(51) Int. Cl. A61B 17/58

(52) U.S. Cl. 606/254; 606/246

(57) ABSTRACT

A flexible rod for fixing vertebrae is connected to a vertebral fixing device in order to maintain a damaged or deformed vertebra in an original stable state and impart flexibility. The flexible rod comprises a plurality of support bars, each of which has an insertion recess that has internal threads on an open side thereof; a plurality of rings, which is disposed between the support bars and has internal threads; a spring, which has a spiral structure so as to be able to be coupled into the support bars in engagement with the threads of the support bars; and an elastic bar, which is inserted into the support bars through the center of the spring.

### BACKGROUND OF THE INVENTION

1. Field of the Invention

The present invention relates to a rod for fixing vertebrae, which is used to help a damaged or deformed vertebra restore fully to an original stable state.

A variety of vertebral fixing devices are used to treat such a vertebra. As one example, the vertebral fixing device comprises pedicle screws and an elastic rod for connecting the pedicle screws. When the vertebral fixing device is installed, the pedicle screws are coupled to respective pedicles, which are intended to be connected in a vertical direction, and the elastic rod for connecting the pedicle screws is fitted into a fastener formed at the upper portion of each pedicle screw, and is then fixed so as to be placed in a fastening recess of the fastener of each pedicle screw. A fixing bolt is fastened to an internally threaded part of the fastener until the bottom thereof presses the elastic rod. Thereby, the elastic rod, connected between the pedicles, is firmly installed.

2. Description of the Prior Art

A known vertebral fixing rod is made of an elastic material, or has a structure in which an elastic connector that imparts elasticity is provided to part of a rectangular elastic rod. This structure will be roughly described below.

In Korean Utility Model Registration No. 338006 (entitled “Elastic Rod for Connecting Pedicle Screws”), the elastic connector of a pedicle screw connecting elastic rod has the shape of a circular ring, part of which is open, in order to solve the problem with a known pedicle screw connecting rod in which it is impossible to move the joint of a bone, as in FIG. 6.

According to Korean Patent Application No. 2006-118258 (entitled “Rod for Connecting Pedicle Screws”), a pedicle fixing device comprises a pedicle screw, which includes both a head having a holding hole having a predetermined depth, which is open in a diametrical direction, perpendicular to an axial direction, in an upper portion thereof and a threaded body extending from the bottom of the head and screwed a predetermined depth into a pedicle, and which serves as an anchor of the pedicle; a rod, which is placed in the holding hole and connects the neighboring pedicle screws to adjust the pedicle angle and distance; and fixing means, which is inserted into the holding hole of the pedicle screw and presses the rod so as to prevent the rod from moving, wherein the rod includes: connecting bars, which have the shape of long cylinders, have elastic restoring force, and are formed on opposite sides thereof; and a leaf spring, which integrally connects the opposite connecting bars and has an elastic part, the width of which is greater than the diameter of each connecting bar, and the thickness of which is less than the diameter of each connecting bar, and a connecting part, which connects the elastic part and the connecting bars so as to form a smooth curve rather than an angled corner, and wherein the connecting bars and the leaf spring are machined by cutting a single material.

According to Korean Patent No. 604992 (entitled “Rod of a Spine Fixation Apparatus”), the rod of a spine fixation apparatus is coupled to pedicle screw members so as to support vertebrae, and includes a plurality of open grooves formed at predetermined intervals in a longitudinal direction such that proper elasticity is exerted without a change in diameter, neighboring open grooves are formed with an angular interval therebetween of 90° in the circumferential direction of the rod such that the rod maintains isotropy, and each open groove has a depth ranging from the surface to the center of the rod, and is provided with a hole, the diameter of which is greater than a width thereof, in the diametrical direction of the rod.

According to Korean Patent No. 766580 (entitled “Rod-like Element for Application in Spinal or Trauma Surgery, and Stabilization Device with such a Rod-like Element”), a rod-like element includes a hollow cylindrical rod, which has a first rigid part, spring parts provided in the proximity of and on opposite sides of the first rigid part, and rigid ends disposed adjacent to the respective spring parts.

As in FIG. 6, the elastic connector of the pedicle screw connecting elastic rod has the shape of a circular ring, part of which is open. When the elastic rod is mounted on the vertebrae in order to fix the vertebrae, the elastic rod having the open circular ring shape has an influence on surrounding tissues and nerves due to a portion that protrudes outwards. The elastic rod is considered to have unsatisfactory industrial applicability.

Further, in order to overcome this drawback, as in FIG. 7, the elastic connector of the elastic rod is provided in a plate shape. This elastic connector allows flexion in forward and backward directions. However, this elastic connector does not allow flexion in a lateral direction.

Thus, most elastic rods have recently been fabricated in the shape of a circular pipe, in which the elastic connector has the shape of a spring.

FIG. 8 shows an embodiment of such an elastic rod having the circular pipe shape. This elastic rod implements a circular structure so as to minimize the area of the elastic connector that protrudes outwards, so that structural simplification and the consequent low cost of production are ensured. Nevertheless, the structure in which flexion is allowed comprises open grooves, formed by cutting out half of the circumference of the cylinder, and holes having a diameter greater than the width of each open groove in the diametrical direction of the rod. Further, the open grooves are formed with an angular difference of 90° therebetween in the circumferential direction of the elastic rod at regular intervals.

Thus, flexion is possible only in the direction corresponding to the position where each open groove is cut out, which means that it is difficult for an elastic rod having open grooves at an interval of 90° to have a strong restoring force. Further, materials in the human body are introduced into the holes, so that the elastic force can be reduced by repeated flexion-extension movement.

In order to improve this problem, as in FIG. 9, the elastic rod has the elastic connector having a spiral structure, so that it can provide a denser flexion-extension range to the space where the flexion acts. However, inflow occurs around an open spiral groove due to the friction of tissues around the acting part. When the inflowing material sticks to and accumulates in the spiral groove, a patient may feel pain, and the flexible range is also reduced.

### SUMMARY OF THE INVENTION

Accordingly, the present invention has been made keeping in mind the above problems occurring in the prior art, and an object of the present invention is to provide an innovative flexible rod for fixing vertebrae, which has good restoring force and durability.

The present invention is directed to a flexible rod for fixing vertebrae, the outer circumference of which is isolated from the inside thereof so as to prevent nervous tissues or materials in the human body from being introduced from the outside thereof through an internal cavity or a gap.

Further, the present invention is directed to a flexible rod for fixing vertebrae, which can prevent foreign materials from being deposited in a gap because the spring, which is inserted into the flexible rod in order to impart elastic force thereto, has no gap between the spirals thereof.

In an aspect, the present invention provides a flexible rod for fixing vertebrae, which is connected to a pedicle fixation device, the pedicle fixation device including a pedicle screw, which has a holding recess having a predetermined depth in an upper portion thereof, and a threaded body driven a predetermined depth into a pedicle and serving as an anchor of the pedicle, and fixing means, which is inserted into the holding recess of the pedicle screw and presses the flexible rod so as to prevent the flexible rod from moving, the flexible rod comprising: a plurality of support bars, each of which has an insertion recess that has internal threads on an open side thereof; a plurality of rings, which is disposed between the support bars and has internal threads; a spring, which has a spiral structure so as to be able to be coupled into the support bars through engagement with the threads of the support bars; and an elastic bar, which is inserted into the support bars through the center of the spring.

As described above, the embodiment of the flexible rod used for the vertebral fixing device having a pedicle screw connection type has been proposed, but is only one embodiment for accomplishing the effects of the present invention. Thus, it is apparent to those skilled in the art that the present invention is not limited to such an embodiment, but can be modified for application to various vertebral fixing devices for providing consistency of action within a range of elastic restoring force and ensuring fixation and activity of the vertebrae so as to minimize friction with surrounding tissues within the scope of the technical spirit of the present invention. It is natural that such modifications fall to the scope and spirit of the invention defined in the accompanying claims in view of the equivalents having substantially the same configuration, purposes and effects as the present invention, so that the industrial applicability of the present invention is very high.

### BRIEF DESCRIPTION OF THE DRAWINGS

The above and other objects, features and other advantages of the present invention will be more clearly understood from the following detailed description when taken in conjunction with the accompanying drawings, in which:

FIG. 1 is a perspective view illustrating the overall structure of a flexible rod according to the present invention;

FIG. 2 is a front view illustrating the operation of a flexible rod according to the present invention;

FIG. 3 is an exploded perspective view illustrating a flexible rod according to the present invention;

FIG. 4 is a sectional view illustrating a flexible rod according to the present invention;

FIG. 5 is a perspective view illustrating the state in which a flexible rod according to the present invention is applied to vertebrae;

FIG. 6 illustrates the typical structure of a known vertebral fixing device as an embodiment for reference;

FIG. 7 illustrates the typical structure of a known vertebral fixing device as another embodiment for reference;

FIG. 8 illustrates the typical structure of a known vertebral fixing device as another embodiment for reference; and

FIG. 9 illustrates the typical structure of a known vertebral fixing device as another embodiment for reference.

### DETAILED DESCRIPTION OF THE INVENTION

Reference will now be made in greater detail to an exemplary embodiment of the invention, an example of which is illustrated in the accompanying drawings. Wherever possible, the same reference numerals will be used throughout the drawings and the description to refer to the same or like parts.

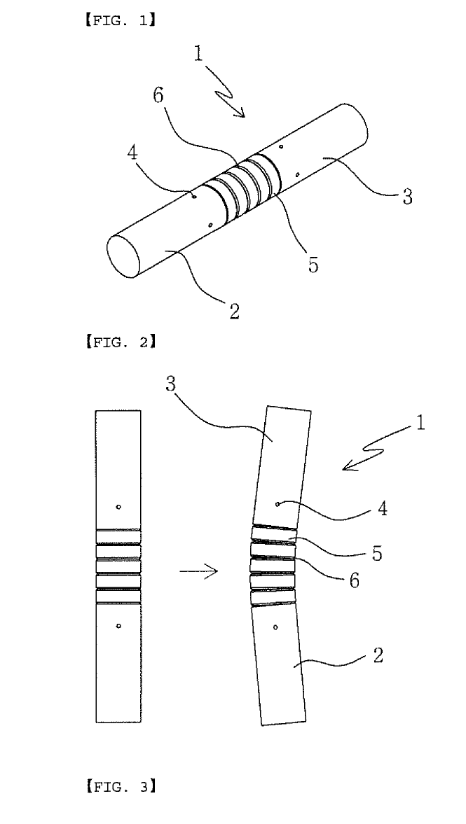

FIGS. 1 and 4 illustrate the overall structure and internal cross section of a flexible rod. The flexible rod, which is connected to a pedicle fixation device, the pedicle fixation device including a pedicle screw, which has a holding recess having a predetermined depth in an upper portion thereof and a threaded body driven a predetermined depth into a pedicle, and serves as an anchor of the pedicle, and a fixing means, which is inserted into the holding recess of the pedicle screw and presses the flexible rod so as to prevent the flexible rod from moving, comprises: a plurality of support bars 2 and 3, each of which has an insertion recess that has internal threads in the open side thereof; a plurality of rings 5, which are disposed between the support bars 2 and 3 and have internal threads 9; a spring 7, which has a spiral structure so as to be able to be coupled into the support bars 2 and 3 in engagement with the threads of the support bars 2 and 3; and an elastic bar 8, which is inserted into the support bars 2 and 3 through the center of the spring 7.

Here, the support bars 2 and 3 are spaced apart from each other in a symmetrical shape, and are coupled to each other by the spring 7 with the rings 5 disposed therebetween. Further, each support bar includes a support hole 11, which has a diameter greater than that of the inserted elastic bar 8 in order to hold the elastic bar 8, and fixing depressions 4 and protrusions 4′, which are formed along an outer circumference at regular intervals by pressing, and prevent the spring 7 from being separated from the respective support bars by external impacts or vibration.

The fixing depressions 4 and protrusions 4′ are formed along the outer circumference of each of the support bars 2 and 3 at an interval of 90°, and thus are four in number. Each ring 5 has chamfers 6 in order to minimize friction and damage to contact ends thereof, caused by flexion-extension movement between the neighboring rings. The elastic bar 8 passes through the spring 7, and is then welded to the spring 7 on opposite ends thereof, and portions where the elastic bar and the spring are welded are finished, and have finished faces.

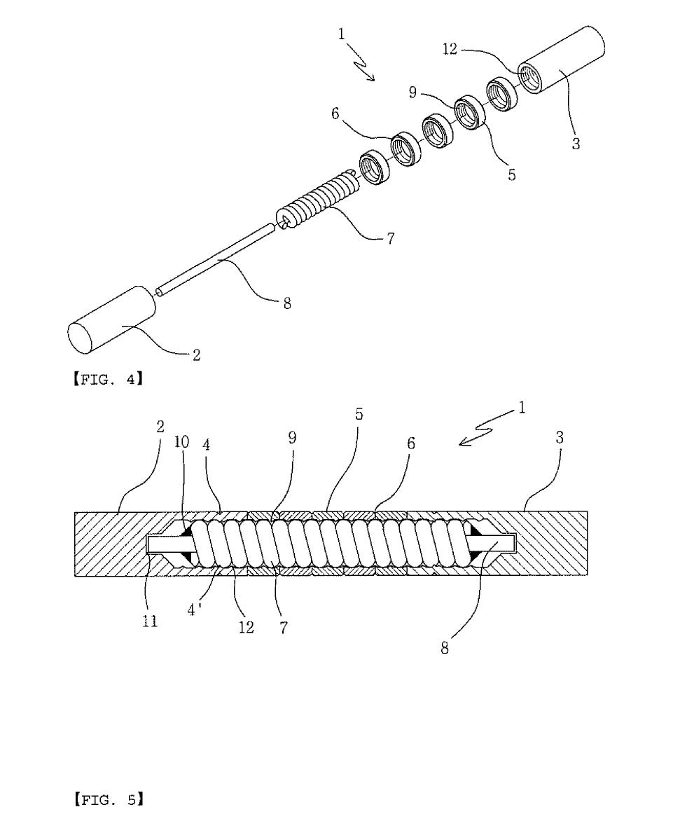

FIG. 2 illustrates the operation of a flexible rod, and FIG. 3 is an exploded perspective view illustrating a flexible rod, As illustrated, the support bar 2 of the flexible rod is made of titanium, and is provided with a coupling recess 12, which is internally threaded on the open side of the support rod 2.

Possible materials for the support bar include, but are not limited to, titanium. Therefore, it is apparent that the material of the support bar can be any material maintaining rigidity and elasticity.

The spring 7, having elasticity, is coupled to the coupling recess 12 of the support bar 2.

The coupled spring has a spiral structure, and is characterized in that neighboring spirals are in close contact with each other without a gap therebetween. Such a gap can cause a problem: human tissues or nerve materials can enter this gap, thus causing pain to a patient at the time of flexion movement, or deform the elastic bar, and furthermore make precise flexion movement impossible.

Thus, as in FIG. 3, the spring 7 is characterized by a spiral, close structure without a gap in order not only to prevent nervous tissues or materials in the human body from being introduced but also to form the threads coupled to the support bars and the rings.

In this manner, when the spring 7 is coupled to the support bar 2, each ring 5, having the threads 9, is turned and coupled along the helix of the spring 7. The number of coupled rings 5 can be freely adjusted according to the features of the patient to whom the vertebral fixation flexible rod 1 is applied.

The spring 7, to which the plurality of rings 5 is coupled, is coupled by the other support bar 3 at the other end thereof.

Further, the elastic bar 8, the shape of which is recommended to be a cylindrical shape, is inserted into the center of the spring 7. Thus, the elastic bar 9 passes through the spring 7 and is welded to the opposite ends of the spring 7.

For the purpose of post-treatment of the welded portions, each welded portion can be finished to form a finished face 10 by removing impurities such as welding spatter or chamfering an angled corner.

Further, the elastic bar 8, which is fixed to the spring 7, is inserted into and fixed to the support holes 11 of the support bars 2 and 3 at the opposite ends of the spring 7.

When the flexible rod 1 is assembled in this way, the regions of the support bars 2 and 3 to which the spring 7 is coupled are pressed using, for instance, a press in order to prevent the spring 7 from being separated from the support bars 2 and 3, so that the fixing depressions 4 are formed. Simultaneously, together with the fixing depressions 4, the fixing protrusions 4′ are formed inside the support bars 2 and 3, thereby pressing the spring 7. Thus, the fixing protrusions 4′ can prevent the spring 7 from being uncoupled from the support bars 2 and 3 by inhibiting the rotation of the spring 7.

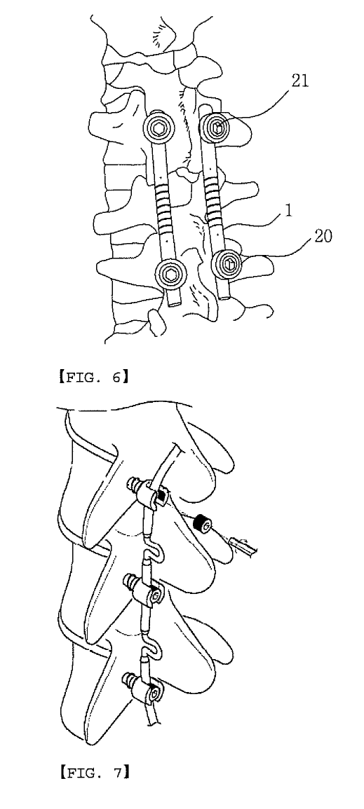

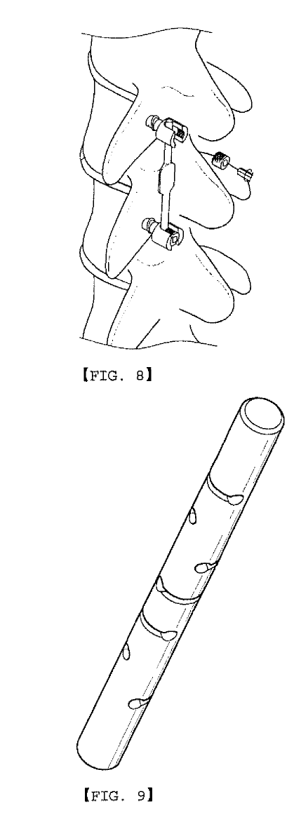

FIG. 5 illustrates the state in which a flexible rod is applied to vertebrae. The example of FIG. 5 shows an example of the general operation thereof, and thus is not intended to limit the applicability thereof.

A variety of vertebral fixing devices are used to treat the vertebrae. As one example, the vertebral fixing device comprises pedicle screws 20 and an elastic rod 1 for connecting the pedicle screws. When the vertebral fixing device is installed, the pedicle screws 20 are coupled to respective pedicles intended to be connected in a vertical direction, and the elastic rod 1 for connecting the pedicle screws is fitted into a fastener formed at an upper position of each pedicle screw, and is then fixed so as to be placed in a fastening recess of the fastener of each pedicle screw. A fixing bolt 21 is fastened to an internally threaded part of the fastener, until the bottom thereof presses the elastic rod 1. Thereby, the elastic rod 1, connected between the pedicles, is firmly installed.

Although an exemplary embodiment of the present invention has been described for illustrative purposes, those skilled in the art will appreciate that various modifications, additions and substitutions are possible, without departing from the scope and spirit of the invention as disclosed in the accompanying claims.

What is claimed is:

1. A flexible rod for fixing vertebrae, which is connected to a pedicle fixation device, the pedicle fixation device including a pedicle screw, which has a holding recess having a predetermined depth in an upper portion thereof, and a threaded body driven a predetermined depth into a pedicle and serving as an anchor of the pedicle, and fixing means, which is inserted into the holding recess of the pedicle screw and presses the flexible rod so as to prevent the flexible rod from moving, the flexible rod comprising:
a plurality of support bars, each of which has an insertion recess that has internal threads on an open side thereof;
a plurality of rings, which is disposed between the support bars and has internal threads;
a spring, which has a spiral structure so as to be able to be coupled into the support bars through engagement with the threads of the support bars; and
an elastic bar, which is inserted into the support bars through the center of the spring.

2. The flexible rod according to claim 1, wherein the support bars are spaced apart from each other in a symmetrical arrangement, and are coupled to each other by the spring, with the rings disposed therebetween.

3. The flexible rod according to claim 1, wherein each of the support bars includes a support hole, which has a diameter greater than that of the inserted elastic bar in order to hold the elastic bar.

4. The flexible rod according to claim 1, wherein each of the support bars, which are coupled to each other by the spring with the rings disposed therebetween, includes fixing depressions and protrusions, which are formed along an outer circumference thereof at regular intervals by pressing, and prevent the spring from being separated from the respective support bars by external impact or vibration.

5. The flexible rod according to claim 2, wherein each of the support bars, which are coupled to each other by the spring with the rings disposed therebetween, includes fixing depressions and protrusions, which are formed along an outer circumference thereof at regular intervals by pressing, and prevent the spring from being separated from the respective support bars by external impact or vibration.

6. The flexible rod according to claim 4, wherein the fixing depressions and protrusions are formed along the outer circumference of each of the support bars at an interval of 90°, and thus are four in number.

7. The flexible rod according to claim 5, wherein the fixing depressions and protrusions are formed along the outer circumference of each of the support bars at an interval of 90°, and thus are four in number.

8. The flexible rod according to claim 1, wherein each ring includes at least one chamfer in order to minimize friction and damage of at least one of opposite ends thereof, which are caused by flexion-extension movement between the neighboring rings.

9. The flexible rod according to claim 1, wherein the elastic bar passes through the spring, and is then welded to the spring on opposite ends thereof, and portions where the elastic bar and the spring are welded are finished, and have finished faces.

10. The flexible rod according to claim 1, wherein the spring is a close spiral structure lacking a gap in order to prevent introduction of surrounding materials.

11. The flexible rod according to claim 1, wherein the rings are changed in number when the flexible rod is fabricated.

12. The flexible rod according to claim 1, wherein the support bars are changed in length according to characteristics of a patient.
